(19) United States
(12) Patent Application Publication
(10) Pub. No.: US 2010/0048760 A1
(43) Pub. Date: Feb. 25, 2010

(54) RAPIDLY DISPERSABLE, PARTICULATE FILM-COATING COMPOSITION BASED ON POLYVINYL ALCOHOL-POLYETHER GRAFT COPOLYMERS

(75) Inventors: Karl Kolter, Limburgerhof (DE); Angelika Maschke, Regensburg (DE); Franz-Josef Dietzen, Hassloch (DE); Thorsten Schmeller, Wachenheim (DE)

Correspondence Address: CONNOLLY BOVE LODGE & HUTZ LLP, 1875 EYE STREET, N.W., SUITE 1100, WASHINGTON, DC, 20006, US

(73) Assignee: BASF SE, Ludwigshafen (DE)

(21) Appl. No.: 12/515,171

(22) PCT Filed: Dec. 11, 2007
(86) PCT No.: PCT/EP07/63674

§ 371 Date: May 15, 2009

(30) Foreign Application Priority Data
Dec. 29, 2006 (EP) 06127337.1

(51) Int. Cl. A61K 8/81; B29B 9/04

(52) U.S. Cl. 523/105; 264/5

(57) ABSTRACT

Solid particulate film-coating compositions consisting of
a) 40-90% by weight, preferably 40 to 70% by weight, of a polyvinyl alcohol-polyether graft copolymer (component A),
b) 10 to 60% by weight, preferably 20 to 45% by weight, of a chromatic or achromatic colorant (component B)
c) 0 to 30% by weight of further conventional auxiliaries (component C)
   where the amount of components A to C adds up to 100% by weight, which are obtained by extrusion of a melt of components A) to C) and subsequent shaping.

The present invention relates to solid rapidly dispersible film-coating compositions for coating pharmaceutical dosage forms or dietary supplements which consist of at least one polyvinyl alcohol-polyether graft copolymer (component A) and further conventional coating constituents, especially colorants. The invention further relates to a process for producing such solid particulate film-coating compositions.

Solid dosage forms are provided with a rapidly dissolving coating for a wide variety of reasons. Thus, for example, it is possible to improve the appearance, the distinguishability and the swallowability, to mask a bitter taste or to protect the dosage form from external influences such as, for example, moisture or oxygen. Since in the case of rapidly releasing dosage forms the film coating is intended to dissolve rapidly in various aqueous media, inter alia in simulated gastric and intestinal fluids, the most important ingredient of the film-coating composition must be a water-soluble, film-forming polymer. The film-forming polymers employed for coating tablets are mainly hydroxypropylmethylcellulose and hydroxypropylcellulose, but they have serious disadvantages. Thus, the viscosity of these polymers in water is very high and permits a concentration of only up to about 10%, because the high viscosity at higher concentrations means that fine atomization in the spray nozzle is no longer possible, and the coating is rough, inhomogeneous and unsightly. In addition, these polymers are very brittle and frequently develop cracks during storage, especially if the core changes in volume through uptake or release of moisture.

The use of polyvinyl alcohol-polyether graft copolymers as coating agents or binders in pharmaceutical dosage forms or as packaging material or as additive in cosmetic, dermatological or sanitary preparations is disclosed for example in WO 00/18375. Thus, for example, a formula for a film-coating composition which consists of a polyvinyl alcohol-polyether graft copolymer and the usual coating ingredients for coloring and opacity, namely iron oxide, talc and titanium dioxide is described. However, the mixtures described therein still left room for improvement.

WO 03/070224 describes coatings consisting of polyvinyl alcohol-polyether graft copolymers, of a component having hydroxy, amide or ester functions and of further usual coating ingredients. In this case there is initial production of a premix of the starting materials as physical mixture, and the latter is then dispersed in water. These preparations are prone to segregation and their asperities are not good.

On the other hand, there is a great interest in finished premixes for film-coating compositions which, besides the film-forming polymer, comprise conventional auxiliaries such as, for example, pigments or further ingredients which do not become segregated, this being important for consistent quality of the coating, are non-dusting and easily storable and can be redispersed simply and rapidly before use as film-coating composition.

WO 06/002808 discloses film-coating compositions in powder form which, besides polyvinyl alcohol-polyether graft copolymers, comprise polyvinylpyrrolidones, pigments and surfactants, which are obtained by grinding the pigments in the presence of aqueous solutions of the polymer and of the other ingredients and subsequent spray drying. However, these film-coating compositions still leave room for improvement in relation to the freedom from dust and ease of handling by the user.

The invention was based on the object of developing a film coat which in solid form does not lead to any segregation between the individual constituents, in particular between pigments and polymers, which has excellent flow characteristics, which can be dissolved very simply and rapidly in water, resulting in a very short time to produce the preparation for spraying, which can be sprayed with high polymer and solids concentrations and with high spraying rate without the spray nozzle becoming blocked, which spreads very well on the surface, which is flexible and forms no cracks during storage, which is not tacky, which adheres well to all surfaces, which exhibits excellent smoothness and gloss, which is very stable to mechanical stress, and which dissolves very rapidly. The object was in particular to find solid film-coating compositions which can be handled easily in relation to freedom from dust and electrostatic charge.

Accordingly, solid particulate film-coating compositions consisting of
a) 40-90% by weight, preferably 40 to 70% by weight, of a polyvinyl alcohol-polyether graft copolymer (component A),
b) 10 to 60% by weight, preferably 20 to 45% by weight, of a chromatic or achromatic colorant (component B)
c) 0 to 30% by weight of further conventional auxiliaries (component C)
   have been found, where the amount of components A to C adds up to 100% by weight, which are obtained by extrusion of a melt of components A) to C) and subsequent shaping.

The film-coating compositions have a narrow particle size distribution, where the average particle sizes (d05, volume average) are in the range from 300 μm to 2000 μm, preferably 500 μm to 1500 μm. The distribution span is <1, preferably <0.8. The distribution span is calculated by the following formula: (d(09)-d(01))/d(05). The film-coating compositions of the invention consist of oval to spheroidal lenticular particles.

The film-coating compositions are rapidly dispersible in the application medium, in particular in water.

The film-coating compositions are obtained by a process which comprises components A) and B) and, if appropriate, C) being processed to a melt, extruded and subjected to shaping.

Polyvinyl alcohol-polyether graft copolymers mean polymers which are obtainable by polymerizing
a) at least one vinyl ester of aliphatic C₁-C₂₄-carboxylic acids preferably vinyl acetate, in the presence of
b) polyethers of the general formula I,

R¹⁻O—(R²⁻O)ₓ₋(R³⁻O)_{y-}(R⁴⁻O)_{z-}R⁵ I
c) in which the variables have independently of one another the following meaning:
   R¹ hydrogen, C₁-C₂₄-alkyl, R⁶—C(═O)—, polyalcohol residue;
      preferably: R₁═H, CH₃—
   R⁵ hydrogen, C₁-C₂₄-alkyl, R⁶—C(═O)—;
      preferably: R⁵═H
   R² to R⁴
      —(CH₂)₂—, —(CH₂)₃—, —(CH₂)₄—, —CH₂—CH(CH₃)—, —CH₂—CH(CH₂—CH₃)—, —CH₂—CHOR⁷—CH₂—;
      preferably R₂ to R₄: —(CH₂)₂—, —CH₂—CH(CH₃)—
      very particularly preferably R₂ to R₄: —(CH₂)₂—
   R₆ C₁-C₂₄-alkyl;
   R₇ hydrogen, C₁-C₂₄-alkyl, R⁶—C(═O)—;
   x 1 to 5000;
      preferably: x=10 to 2000;
      very particularly preferably: x=20 to 500
   y 0 to 5000;
      preferably: y=0
   z 0 to 5000;
      preferably: z=0,
   with the proviso that x≧10 when y and z=0,
      and subsequent complete or partial hydrolysis of the polyvinyl ester groups.
   x, y, z:
   calculation of the molecular weight of the polyether from x, y and z results in an average, because corresponding products usually have a broad distribution of molecular weight.

Preferred polyethers have an average molecular weight between 400 and 50 000 g/mol, particularly preferably from 1500 to 20 000 g/mol.

The preparation of such graft copolymers is known per se and is described for example in WO 00/18357, which is incorporated herein by reference.

Preferred polymers have a degree of hydrolysis of the polyvinyl ester groups of >70 mol %, particularly preferably >80 mol % and very particularly preferably of >85 mol %.

A particularly preferred polyvinyl alcohol-polyether graft copolymer is one in which
a) vinyl acetate has been used as monomer for grafting,
b) the variables have the following meaning:
   R¹═H
   R²—R⁴═—(CH₂)₂₋
   R₅═H
   x=20 to 500
   y=0
   z=0
   and thus represent a polyethylene glycol with an average molecular weight of 6000
c) the degree of hydrolysis of the ester groups is >85 mol %, and
d) the mass ratio of the polyvinyl alcohol/polyethylene glycol 6000 moieties is 75:25.

In addition, the film-coating compositions comprise as components B colorants, in particular organic or inorganic pigments. Colorants which can be employed are chromatic or achromatic colorants, the meaning of achromatic agents according to the invention being white, grey or black agents, preferably white agents, in particular white pigments.

Pigments refer to coloring or white substances which are insoluble in the application medium.

The pigments can be fed into the process of the invention in pure form or as pigment preparations. Such pigment preparations are known to the skilled worker and, just like the pure pigments, are commercially available.

Suitable inorganic pigments are aluminum silicates, magnesium silicates, magnesium-aluminum silicates, iron oxide, titanium dioxide, zinc oxide, silica, or calcium hydrogen phosphate. Of the aluminum silicates, kaolin is particularly suitable. Of the magnesium silicates, talc is particularly important.

Preferred pigments are iron oxide or iron oxide preparations, for example the commercially available Sicovit® Yellow 10 E172 or Sicovit® Red 30 E172, and white pigments selected from the group consisting of titanium dioxide, talc and kaolin.

Suitable organic pigments are organic lakes or mixtures thereof. Examples of organic lakes which can be used are: carmine lake, quinoline yellow lake, tartrazine lake, orange-yellow lake, FD&C yellow aluminum lake, cochineal red lake, erythrosine lake, azorubine lake, indigotine lake, brilliant blue, beta-carotene.

The film coatings may further comprise as components C if appropriate up to 30% by weight of auxiliaries as are customary as coating constituents. Further customary coating constituents comprise:

surfactants, water-soluble dyes, non-stick agents, polymeric binders, fillers, swelling agents, gloss improvers, antifoams, protective colloids, buffer substances, pH-regulating substances, bonding agents or plasticizers.

Substances which can be employed as surfactants have an HLB (Hydrophilic Lipophilic Balance; cf. Fiedler, Lexikon der Hilfsstoffe, Editio Cantor Verlag Aulendorf, 5^{th} edition (2002), pages 1 15-121) greater than 10.

Those particularly suitable are alkali metal salts of C8-C30 fatty acids, C8-C30 alkylsulfonates, C8-C30-alkyl sulfates, C8-C30-alkylarylsulfonates or dioctyl sulfosuccinate, ethoxylates of C8-C30-fatty acids, C8-C30-fatty alcohols, fatty acid glycerides, sorbitan fatty acid esters, sorbitan fatty alcohol ethers or phenols, and polyoxypropylene-polyoxyethylene block copolymers. Examples from the classes of substances mentioned are sodium stearate, sodium oleate, sodium laurylsulfonate, sodium lauryl sulfate, polyoxyethylene (9) monostearate, polyoxyethylene (10) stearyl cetyl ether, polysorbate 80, polysorbate 20, ethoxylated castor oil (35 EO), ethoxylated hydrogenated castor oil (40 EO), ethoxylated 1 2-hydroxystearic acid (15 EO), poloxamer 188, poloxamer 408.

Examples of suitable fillers are celluloses such as microcrystalline cellulose, also sugars such as lactose, sucrose or dextrose, sugar alcohols such as mannitol, sorbitol, xylitol or isomalt.

Suitable swelling agents are crospovidones, croscarmellose, sodium carboxymethyl starch or uncrosslinked carboxymethylcellulose.

Suitable polymeric binders are homo- and copolymers of N-vinylpyrrolidones, for example povidone having Fikentscher K values of from 12 to 90 or copovidone.

The solid dosage forms are produced by preparing a plastic mixture of the components, which is subsequently subjected to a shaping step. The mixing of the components and the plastication of the mixture can take place in various ways.

Plastication means a softening of the mixture by the action of pressure, shear forces, temperature and/or plasticizers. The softening preferably takes place in the sense of achieving thermoplasticity by the combined action of raising the temperature and shear forces, in particular in combination with plasticizers.

Suitable plasticizers are long-chain alcohols, ethylene glycol, propylene glycol, glycerol, trimethylolpropane, triethylene glycol, butanediols, pentanols such as pentaerythritol, hexanols, polyethylene glycols, polypropylene glycols, polyethylene-propylene glycols, silicones, aromatic carboxylic esters (e.g. dialkyl phthalates, trimellitic esters, benzoic esters, terephthalic esters) or aliphatic dicarboxylic esters (e.g. dialkyl adipates, sebacic esters, azelaic esters, citric and tartaric esters, especially triethyl citrate, fatty acid esters, glycerol mono-, di- or triacetate or sodium diethyl sulfosuccinate or mixtures of said plasticizers. Water is also suitable and is preferred as plasticizer.

The amount of plasticizer is sufficient for the mass to remain plastic and extrudable. The amount added will normally not exceed 30% by weight based on the total of the amount of components A to C.

A further possibility is also to employ pore-forming agents during the processing. Such agents may lead to foaming during extrusion or, through the formation of the pores during the dissolution of the finished coating, to accelerated disintegration. Suitable as such pore formers are carbonates or bicarbonates of alkali metals or alkaline earth metals such as, for example, sodium, potassium, magnesium or calcium or the corresponding ammonium compounds. It is also possible to employ gaseous carbon dioxide. A further possibility is also to add acids, especially organic acids such as citric acid, tartaric acid, lactic acid, maleic acid, fumaric acid or succinic acid.

The mixing of the components can take place before, during and/or after formation of the melt. For example, the components can first be mixed and then plasticated or be mixed simultaneously and then plasticated. The plastic mixture is frequently also homogenized in order to obtain a highly dispersed distribution of the colorant.

The components are generally employed as such in the production process. However, they can also be used in liquid form, i.e. as solution, suspension or dispersion.

Suitable solvents for the liquid form of the components are primarily water or a water-miscible organic solvent or a mixture thereof with water. However, it is also possible to use organic solvents which are immiscible or miscible with water. Suitable water-miscible solvents are, in particular, C₁-C₄-alkanols such as ethanol, isopropanol or n-propanol, polyols such as ethylene glycol, glycerol and polyethylene glycols. Suitable water-immiscible solvents are alkanes such as pentane or hexane, esters such as ethyl acetate or butyl acetate, chlorinated hydrocarbons such as methylene chloride, and aromatic hydrocarbons such as toluene and xylene. Another solvent which can be used is liquid CO₂. The solvents may also simultaneously serve as plasticizers.

The solvent used in the individual case depends on the composition to be processed. The amount of solvent must be limited in every case so that the composition to be processed remains plastic and extrudable.

The plastication and/or mixing takes place in an apparatus customary for this purpose. Particularly suitable ones are extruders or containers which can be heated where appropriate and have an agitator, e.g. kneaders (like those of the type to be mentioned below).

A particularly suitable mixing apparatus is one employed for mixing in plastics technology. Suitable apparatuses are described, for example, in “Mischen beim Herstellen und Verarbeiten von Kunststoffen”, H. Pahl, VDI-Verlag, 1986. Particularly suitable mixing apparatuses are extruders and dynamic and static mixers, and stirred vessels, single-shaft stirrers with stripper mechanisms, especially paste mixers, multishaft stirrers, especially PDSM mixers, solids mixers and, preferably, mixer/kneader reactors (e.g. ORP, CRP, AP, DTB supplied by List or Reactotherm supplied by Krauss-Maffei or Ko-Kneader supplied by Buss), trough mixers and internal mixers or rotor/stator systems (e.g. Dispax supplied by IKA).

The mixing apparatus is charged continuously or batchwise, depending on its design, in a conventional way. Powdered components can be introduced in a free feed, e.g. via a weigh feeder. Melts can be fed in directly from an extruder or via a gear pump, which is particularly advantageous if the viscosities and pressures are high. Liquid media can be metered in by a suitable pump unit.

The mixture obtained by mixing and/or softening the film-coating composition and, where appropriate, the additive(s) ranges from pasty to viscous (plastic) or fluid and is therefore extrudable. The glass transition temperature of the mixture is below the decomposition temperature of all the components present in the mixture.

The steps of mixing and plasticating in the process can be carried out in the same apparatus or in two or more separately operating apparatuses. The preparation of a premix can take place in one of the conventional mixing apparatuses described above. A premix of this type can then be fed directly, for example, into an extruder and subsequently extruded, where appropriate with the addition of other components.

It is possible in the novel process to employ as extruders single screw machines, intermeshing screw machines or else multiscrew extruders, especially twin screw extruders, corotating or counterrotating and, where appropriate, equipped with kneading disks. If it is necessary in the extrusion to evaporate a solvent, the extruders are generally equipped with an evaporating section. Twin screw extruders are particularly preferred.

The process of the invention is normally carried out by plastication at elevated temperature, preferably at temperatures of from 50 to 160 (temperature of the plastic mixture), particularly preferably 75 to 120° C.

The design of the die depends on the polymeric binder used and the required pharmaceutical form.

The plastic mixture is, as a rule, subjected to final shaping. This can result in a large number of shapes depending on the die and mode of shaping. Other shapes can be obtained by extrusion and hot- or cold-cut of the extrudate, for example small-particle and uniformly shaped pellets. Hot-cut pelletization preferably results in lenticular forms.

Owing to their freedom from dust, their uniform particle size distribution, their high bulk density, their good flow characteristics and their form, the film-coating compositions of the invention exhibit particularly user-friendly properties. They cannot only be redispersed easily but also easily handled otherwise and are advantageous in relation to the low electrostatic chargeability.

The bulk density of the film-coating compositions of the invention is greater than 0.4, preferably greater than 0.5 and particularly preferably greater than 0.6 g/ml.

The film-coating compositions of the invention are suitable in principle as film-coating compositions for all dosage forms of bioactive substances. Dosage forms mean here all forms suitable for use as drugs, plant treatment compositions, human and animal foods and for delivering fragrances and perfume oils or other cosmetic active ingredients. These include for example tablets of any shape, pellets or granules. A further possibility is also to coat hard or soft capsules.

Such Dosage Forms Generally Comprise:
I 0.1 to 99% by weight, in particular 0.1 to 60% by weight (based on the total weight of the dosage form), of an active ingredient,
II 1 to 99.9% by weight, in particular 20 to 99% by weight, of a binder or a filler and
III 0 to 30% by weight of further additives,
   where the amounts of I, II and III add up to 100% by weight.

Examples of Suitable Binders are:

Polyvinylpyrrolidone (PVP), copolymers of N-vinylpyrrolidone (NVP) and vinyl esters, in particular vinyl acetate, copolymers of vinyl acetate and crotonic acid, partially hydrolyzed polyvinyl acetate, polyvinyl alcohol, polyvinyl alcohol-polyether graft copolymers, poly(hydroxyalkyl acrylates), poly(hydroxyalkyl methacrylates), polyacrylates and polymethacrylates (Eudragit types), copolymers of methyl methacrylate and acrylic acid, polyacrylamides, polyethylene glycols, cellulose esters, cellulose ethers, especially methyl cellulose and ethyl cellulose, hydroxyalkylcelluloses, especially hydroxypropylcellulose, hydroxyalkylalkylcelluloses, especially hydroxypropylethylcellulose, cellulose phthalates, especially cellulose acetate phthalate and hydroxypropylmethylcellulose phthalate, and mannans, especially galactomannans. Of these, polyvinylpyrrolidone, copolymers of N-vinylpyrrolidone and vinyl esters, poly(hydroxyalkyl acrylates), poly(hydroxyalkyl methacrylates), polyacrylates, polymethacrylates, alkylcelluloses and hydroxyalkylcelluloses are particularly preferred.

The dosage forms may further also comprise plasticizers such as long-chain alcohols, ethylene glycol, propylene glycol, glycerol, trimethylolpropane, triethylene glycol, butanediols, pentanols such as pentaerythritol, hexanols, polyethylene glycols, polypropylene glycols, polyethylene/propylene glycols, silicones, aromatic carboxylic esters (e.g. dialkyl phthalates, trimellitic esters, benzoic esters, terephthalic esters) or aliphatic dicarboxylic esters (e.g. dialkyl adipates, sebacic esters, azelaic esters, citric and tartaric esters, especially triethyl citrate, fatty acid esters such as glycerol mono-, di- or triacetate or sodium diethyl sulfosuccinate, or mixtures of said plasticizers. The concentration of plasticizer is generally from 0.5 to 15, preferably 0.5 to 10, % of the total weight of the mixture.

Conventional pharmaceutical fillers are silicates or diatomaceous earth, dicalcium phosphate, calcium carbonate, magnesium oxide, aluminum oxide, talc, sucrose, glucose, lactose, sugar alcohols such as sorbitol, mannitol, xylitol, maltitol, isomalt, cereal or corn starch, potato starch, microcrystalline cellulose.

Conventional lubricants are aluminum and calcium stearates, talc and silicones, and may be present in a concentration of from 0.1 to 5, preferably 0.1 to 3, % of the total weight of the mixture, likewise also animal or vegetable fats, especially in hydrogenated form and those which are solid at room temperature. These fats preferably have a melting point of 50° C. or above. Triglycerides of C₁₂, C₁₄, C₁₆ and C₁₈ fatty acids are preferred. It is also possible to use waxes such as camauba wax. These fats and waxes may be admixed advantageously alone or together with mono- and/or diglycerides or phosphatides, especially lecithin. The mono- and diglycerides are preferably derived from the abovementioned fatty acid types.

Stabilizers such as antioxidants, light stabilizers, hydroperoxide destroyers, radical scavengers, stabilizers against microbial attack.

It is also possible to add wetting agents, preservatives, release-slowing agents, disintegrants, adsorbents, mold release agents and blowing agents (cf., for example, H. Sucker et al., Pharmazeutische Technologie, Thieme-Verlag, Stuttgart 1978).

The dosage forms to be coated may also comprise substances for producing a solid solution of the active ingredient. Examples of these auxiliaries are pentaerythritol and pentaerythritol tetraacetate, polymers such as polyethylene oxides and polypropylene oxides and their block copolymers (poloxamers), phosphatides such as lecithin, homo- and copolymers of vinylpyrrolidone, surfactants such as polyoxyethylene 40 stearate, and citric and succinic acids, bile acids, sterols and others as indicated, for example, in J. L. Ford, Pharm. Acta Helv. 61 (1986) 69-88.

Auxiliaries are also regarded as being bases and acids added to control the solubility of an active ingredient (see, for example, K. Thoma et al., Pharm. Ind. 51 (1989) 98-101).

The dosage forms to be coated can be produced by granulation, crystallization, compaction, compression, extrusion, solidification or encapsulation.

Active ingredients mean for the purpose of the invention all substances with a physiological effect as long as they do not decompose under the processing conditions. These are, in particular, pharmaceutical active ingredients (for humans and animals), active ingredients for plant treatment, insecticides, active ingredients for human and animal foods, fragrances and perfume oils. The amount of active ingredient per dose unit and the concentration may vary within wide limits depending on the activity and the release rate. The only condition is that they suffice to achieve the desired effect. Thus, the concentration of active ingredient can be in the range from 0.1 to 95, preferably from 0.5 to 80, in particular 1 to 70, % by weight. It is also possible to employ combinations of active ingredients. Active ingredients for the purpose of the invention also include vitamins and minerals. The vitamins include the vitamins of the A group, the B group, by which are meant besides B₁, B₂, B₆ and B₁₂ and nicotinic acid and nicotinamide also compounds with vitamin B properties such as adenine, choline, pantothenic acid, biotin, adenylic acid, folic acid, orotic acid, pangamic acid, carnitine, p-aminobenzoic acid, myo-inositol and lipoic acid, and vitamin C, vitamins of the D group, E group, F group, H group, I and J groups, K group and P group. Active ingredients for the purpose of the invention also include therapeutic peptides. Plant treatment agents include, for example, vinclozolin, epoxiconazole and quinmerac.

The film-coating compositions of the invention are suitable for example for coating dosage forms of the following active ingredients:

acebutolol, acetylcysteine, acetylsalicylic acid, acyclovir, alfacalcidol, allantoin, allopurinol, alprazolam, ambroxol, amikacin, amiloride, aminoacetic acid, amiodarone, amitriptyline, amlodipine, amoxicillin, ampicillin, ascorbic acid, aspartame, astemizole, atenolol, beclomethasone, benserazide, benzalkonium hydrochloride, benzocaine, benzoic acid, betamethasone, bezafibrate, biotin, biperiden, bisoprolol, bromazepam, bromhexine, bromocriptine, budesonide, bufexamac, buflomedil, buspirone, caffeine, camphor, captopril, carbamazepine, carbidopa, carboplatin, cefachlor, cefalexin, cefadroxil, cefazoline, cefixime, cefotaxime, ceftazidime, ceftriaxone, cefuroxime, chloramphenicol, chlorhexidine, chlorpheniramine, chlortalidone, choline, cyclosporin, cilastatin, cimetidine, ciprofloxacin, cisapride, cisplatin, clarithromycin, clavulanic acid, clomipramine, clonazepam, clonidine, clotrimazole, codeine, cholestyramine, cromoglycic acid, cyanocobalamin, cyproterone, desogestrel, dexamethasone, dexpanthenol, dextromethorphan, dextropropoxiphene, diazepam, diclofenac, digoxin, dihydrocodeine, dihydroergotamine, dihydroergotoxin, diltiazem, diphenhydramine, dipyridamole, dipyrone, disopyramide, domperidone, dopamine, doxycycline, enalapril, ephedrine, epinephrine, ergocalciferol, ergotamine, erythromycin, estradiol, ethinylestradiol, etoposide, Eucalyptus Globulus, famotidine, felodipine, fenofibrate, fenoterol, fentanyl, flavin mononucleotide, fluconazole, flunarizine, fluorouracil, fluoxetine, flurbiprofen, folinic acid, furosemide, gallopamil, gemfibrozil, gentamicin, Gingko Biloba, glibenclamide, glipizide, clozapine, Glycyrrhiza glabra, griseofulvin, guaifenesin, haloperidol, heparin, hyaluronic acid, hydrochlorothiazide, hydrocodone, hydrocortisone, hydromorphone, ipratropium hydroxide, ibuprofen, imipenem, imipramine, indomethacin, iohexol, iopamidol, isosorbide dinitrate, isosorbide mononitrate, isotretinoin, itraconazole, ketotifen, ketoconazole, ketoprofen, ketorolac, labetalol, lactulose, lecithin, levocarnitine, levodopa, levoglutamide, levonorgestrel, levothyroxine, lidocaine, lipase, lisinopril, loperamide, lorazepam, lovastatin, medroxyprogesterone, menthol, methotrexate, methyidopa, methylprednisolone, metoclopramide, metoprolol, miconazole, midazolam, minocycline, minoxidil, misoprostol, morphine, multivitamin mixtures or combinations and mineral salts, N-methylephedrine, naftidrofuryl, naproxen, neomycin, nicardipine, nicergoline, nicotinamide, nicotine, nicotinic acid, nifedipine, nimodipine, nitrazepam, nitrendipine, nizatidine, norethisterone, norfloxacin, norgestrel, nortriptyline, nystatin, ofloxacin, omeprazole, ondansetron, pancreatin, panthenol, pantothenic acid, paracetamol, penicillin G, penicillin V, phenobarbital, pentoxifylline, phenoxymethylpenicillin, phenylephrine, phenylpropanolamine, phenytoin, piroxicam, polymyxin B, povidone-iodine, pravastatin, prazepam, prazosin, prednisolone, prednisone, propafenone, propranolol, proxyphylline, pseudoephedrine, pyridoxine, quinidine, ramipril, ranitidine, reserpine, retinol, riboflavin, rifampicin, rutoside, saccharin, salbutamol, salcatonin, salicylic acid, selegiline, simvastatin, somatropin, sotalol, spironolactone, sucralfate, sulbactam, sulfamethoxazole, sulfasalazine, sulpiride, tamoxifen, trandolapril, tegafur, teprenone, terazosin, terbutaline, terfenadine, tetracycline, theophylline, thiamine, ticlopidine, timolol, tranexamic acid, tretinoin, triamcinolone acetonide, triamterene, trimethoprim, troxerutin, uracil, valproic acid, vancomycin, verapamil, vitamin E, zidovudine.

The film-coating compositions are also suitable according to the invention for dosage forms which may be produced as multilayer pharmaceutical forms by coextrusion, in which case a plurality of mixtures of the components described above is fed together to an extrusion die so as to result in the required layered structure of the multilayer pharmaceutical form. It is preferable to use different binders for different layers. Multistage release profiles can be adjusted in this way.

Multilayer drug forms preferably comprise two or three layers. They may be in open or closed form, in particular as open or closed multilayer tablets. At least one of the layers comprises at least one pharmaceutical active ingredient. It is also possible for another active ingredient to be present in another layer. This has the advantage that two mutually incompatible active ingredients can be processed or that the release characteristics of the active ingredient can be controlled. The shaping takes place by coextrusion with the mixtures from the individual extruders or other units being fed into a common coextrusion die and extruded. The shape of the coextrusion dies depends on the required pharmaceutical form. Examples of suitable dies are those with a flat orifice, called a slit die, and dies with an annular orifice.

In specific cases there may be formation of solid solutions. The term solid solutions is familiar to the skilled worker, for example from the literature cited at the outset. In solid solutions of active ingredients in polymers, the active ingredient is in the form of a molecular dispersion in the polymer.

It was surprisingly possible for the graft copolymers to be processed by the process of the invention to give film-coating compositions without crosslinking occurring. On the basis of the structure of the graft copolymers, a skilled worker would have expected crosslinking with elimination of water on exposure to temperature and shear forces.

The following examples are intended to illustrate the invention without, however, restricting it.

### EXAMPLES

General Method

A corotating ZSK 25/1 twin-screw extruder from Werner & Pfleiderer with a screw diameter of 25 mm was used as extruder. The extruder was provided with a feed device and a degassing device and was operated with 8 different temperature zones. The mixture of graft copolymer and components C was vigorously mixed and kneaded in the first three zones at 85-90° C., conveyed into zone 4 and mixed with the pigments in zone 5. The melt was then conveyed into zones 6 to 8 at 100° C. or at 110° C. and then extruded.

The melt was extruded through breaker plates. Die plates with an orifice diameter of 1 mm or 0.8 mm were used. The screw speed was from 100 to 300 rpm with a throughput of from 1.5 to 3 kg/h. The emerging extrudates were cut to shape with a rotating knife, and the resulting particles were dried at 25-40° C. in a vacuum drying oven for 12 h.

The graft polymer employed was a polymer of 75% by weight polyvinyl alcohol units and 25% by weight polyethylene glycol as grafting base (PEG 6000) with a molecular weight in the region of 45 000 daltons and a degree of hydrolysis of 94 mol %. All the Sicovit® types employed (iron oxides: Sicovit Yellow 10E172, Sicovit Red 30 E172; Sicovit Indigotine lake) are commercially available (from BASF Aktiengesellschaft).

Formulations with the compositions detailed below were processed in this way. The data in % relate to % by weight.

Formulation No.

| | 1 | 2 | 3 | 4 |
|---|---|---|---|---|
| Graft copolymer | 65% | 55% | 70% | 65% |
| Titanium dioxide | 29% | 38% | 25% | 15% |
| Colored pigment*⁾ | 6% | 8% | 5% | 6% |
| Kaolin | | | | 15% |

*⁾1a) Sicovit Red, 1b) Sicovit Yellow, 1c) Indigotine lake, 1d) Brilliant blue; formulations Nos. 2-4: Sicovit Red

Particle size distribution (density distribution) formulation No. 1a): d(01) 684 μm, d (05) 927 μm and d(09) 1238 μm, distribution span 0.591, measured with a Camsizer from Retsch Technology GmbH.

| | Formulation No. | | |
|---|---|---|---|
| | 5 | 6 | 7 |
| Graft copolymer | 55% | 52% | 61% |
| Crospovidone | 0 | 2% | 4% |
| Poloxamer 68 | 15% | 15% | 0% |
| Sicovit Red or | 5% | 5% | 6% |
| Indigotine lake | | | |
| Titanium dioxide | 25% | 25% | 29% |

| | Formulation No. | | | | | | | | | |
|---|---|---|---|---|---|---|---|---|---|---|
| | 8 | 9 | 10 | 11 | 12 | 13 | 14 | 15 a/b | 16 | 17 |
| Graft copolymer | 63% | 43% | 55% | 60% | 50% | 60% | 55% | 50/55% | 50% | 60% |
| VA 64 | | 10% | 10% | | | | | | | |
| Copovidone | | | | | | | | | | |
| Colloidal silicon | | | | 5% | | | | | | |
| dioxide | | | | | | | | | | |
| Mannitol | | | | | 15% | | | | | |
| Avicel PH105 | | | | | | 5% | 10% | | | |
| (microcrystalline | | | | | | | | | | |
| cellulose) | | | | | | | | | | |
| PEG 1500 | | | | | | | | 10/15% | | |
| Isomalt | | | | | | | | | 15% | |
| Na bicarbonate | | | | | | | | | | 5% |
| Na lauryl sulfate | 2% | 2% | | | | | | | | |
| Titanium dioxide | 29% | 38% | 29% | 29% | 29% | 29% | 29% | 29% | 29% | 29% |
| Sicovit Red | 6% | 8% | 6% | 6% | 6% | 6% | 6% | 6% | 6% | 6% |

The extrudates and water were mixed with a mixing ratio of 20% by weight extrudate and 80% by weight water. The extrudates were completely dispersed within 40 min.

Determination of the Dust Number:

The optical concentration of the dusting fraction was determined by dispersion in air after free fall in a down pipe and impact on the base of the container. 30 g of a sample are introduced into the down pipe by opening a flap. The dust density is determined by measuring the attenuation of the light of a laser beam (wavelength 670 nm) by the resulting cloud of dust, determining the extinction 0.5 s after opening the flap (maximum value) and 30 s after opening the flap. Addition of the maximum value and 30 s value gives the dust number.

The extrudates of the invention show dust numbers of 3.8 (in each case as average of 3 samples). For comparison, the dust value of a film-coating composition according to Example 2 of WO 06/002808 was measured. This film-coating composition was obtained as described in this example by spray drying an aqueous dispersion of 61% graft copolymer, 7% VA 64, 16% kaolin, 14% titanium dioxide and 2% Na lauryl sulfate. The dust value was 22.3.

Determination of the Electrostatic Charging:

A polyethylene bag (25×40 cm) was electrostatically charged by friction for 1 min and weighed, and 40 g of extrudates were weighed in. The amount weighed in was then emptied out, and the bag was reweighed. On average, 0.24% by weight, based on the amount weighed in, of extrudate remains in the bag.

For comparison, once again the abovementioned film-coating composition was investigated. In this case, 3% by weight of the amount weighed in remained in the bag.

1. A solid particulate film-coating composition consisting of
a) 40-90% by weight, preferably 40 to 70% by weight, of a polyvinyl alcohol-polyether graft copolymer (component A),
b) 10 to 60% by weight, preferably 20 to 45% by weight, of a chromatic or achromatic colorant (component B)
c) 0 to 30% by weight of further conventional auxiliaries (component C)
where the amount of components A to C adds up to 100% by weight, which are obtained by extrusion of a melt of components A) to C) and subsequent shaping.

2. The film-coating composition according to claim 1, comprising as component A a graft copolymer having a mass ratio of the polyvinyl alcohol to polyethylene glycol moieties of 75:25, with a molecular weight of the polyethylene glycol of 6000 daltons.

3. The film-coating composition according to claim 1, comprising as component B inorganic or organic pigments and dyes or mixtures thereof.

4. The film-coating composition according to claim 1, comprising as achromatic colorant a white pigment.

5. The film-coating composition according to claim 1, comprising as components C plasticizers, surfactants, water-soluble dyes, non-stick agents, polymeric binders, fillers, swelling agents, gloss improvers, antifoams, protective colloids, buffer substances, pH-regulating substances or bonding agents.

6. The film-coating composition according to claim 1, comprising as component B organic lakes.

7. The film-coating composition according to claim 1, comprising as component B iron oxide, titanium dioxide, kaolin, talc or mixtures thereof.

8. The film-coating composition according to claim 1, comprising as component C) binders from the group of homo- and copolymers of N-vinylpyrrolidone.

9. The film-coating composition according to claim 1, comprising as component C a plasticizer selected from the group consisting of propylene glycol, polyethylene glycols, polypropylene glycols and polyethylene glycol-polypropylene glycol block copolymers.

10. The film-coating composition according to claim 1, comprising as component C a filler from the group comprising sugars and sugar alcohols.

11. The film-coating composition according to claim 1, comprising microcrystalline cellulose as component C.

12. The film-coating composition according to claim 1, comprising as component C sodium lauryl sulfate.

13. The film-coating composition according to claim 1, comprising as component C a colloidal silicon dioxide.

14. The film-coating composition according to claim 1, obtainable by use of a pore former.

15. The film-coating composition according to claim 1, obtainable via use of a compound selected from the group consisting of carbonates and bicarbonates of the alkali metals or alkaline earth metals and citric acid as pore former.

16. The film-coating composition according to claim 1, having average particle sizes of between 500 and 1500 μm.

17. A process for producing a film-coating composition according to claim 1, which comprises components A) to C) being processed by the action of shear forces to a melt and extruded, and the extrudate being subjected to a shaping to disc-like particles.

18. The process according to claim 17, wherein the production of the melt takes place in the presence of water.

19. The film-coating composition according to claim 2, comprising as component B inorganic or organic pigments and dyes or mixtures thereof.

20. The film-coating composition according to claim 2, comprising as achromatic colorant a white pigment.
